(19) United States
(12) Patent Application Publication
(10) Pub. No.: US 2021/0386415 A1
(43) Pub. Date: Dec. 16, 2021

(54) MEDICAL MATERIAL

(71) Applicant: GUNZE LIMITED, Ayabe-shi, Kyoto (JP)

(72) Inventors: Saki OKUMURA, Ayabe-shi (JP); Yuki SAKAMOTO, Ayabe-shi (JP); Hideki SATO, Ayabe-shi (JP)

(73) Assignee: GUNZE LIMITED, Ayabe-shi, Kyoto (JP)

(21) Appl. No.: 17/287,154

(22) PCT Filed: Mar. 26, 2019
(86) PCT No.: PCT/JP2019/012748

§ 371 Date: Apr. 21, 2021

(30) Foreign Application Priority Data
Nov. 15, 2018 (JP) 2018-214492

(51) Int. Cl. A61B 17/00

(52) U.S. Cl. CPC A61B 17/0057; A61B 2017/00004; A61B 2017/00606; A61B 2017/00592

(57) ABSTRACT

A defect hole closing material achieves low invasive treatment for an atrial septal defect with almost no fear of long-term failure. The defect hole closing material is formed by two cylindrical bodies (a first cylinder part and a second cylinder part) having stitch-like structures of a bioabsorbable material, has a sandglass shape, and includes a coil spring both ends of which are respectively engaged with a first end part and a second end part and is passed through inner parts of the first cylinder part and the second cylinder part from a side of the first end part to a side of the second end part via a substantially central part. An outer cylindrical body is provided at an outermost layer so as to cover a whole of the two cylindrical bodies from an outer side, the outer cylindrical body is a cylinder-shaped body obtained by knitting a bioabsorbable material, and both ends thereof are respectively joined to both ends of the two cylindrical bodies.

### TECHNICAL FIELD

The present invention relates to a medical material for treating a defect hole formed in a biological tissue, and in particular, to a medical material that is set in a catheter, sent to a treatment site through a blood vessel, and placed in a living body.

### BACKGROUND ART

The heart of a human is divided into left and right chambers by a tissue called septum, in which each of the left and the right chambers has an atrium and a ventricle. That is, the heart is configured by two atria and two ventricles, i.e., a right atrium, a right ventricle, a left atrium, and a left ventricle. As for the heart having such a configuration, there is known an atrial septal defect (ASD) that is a disorder caused by developmental difficulty in a fetal stage, wherein a hole called a defect hole congenitally opens in an atrial septum partitioning the right atrium and the left atrium.

As treatment for the atrial septal defect, the following two methods exist. One is a surgical operation that is performed with chest cut, and the other one is catheterization using an occluder without cutting the chest.

The surgical operation (patch operation) uses a cardiopulmonary bypass, opens the chest, and closes the defect hole by a patch. The catheterization sets the occluder in a catheter, inserts the catheter into a blood vessel, sends the catheter to a target position (defect hole), and then, releases the occluder to place it in the body. The catheterization is performed in such a manner that without incising the chest, a small jig (device) called the occluder, which is folded into an elongated shape, is sent from a vein (femoral vein) at the root of a leg to a position of the hole opening in the atrial septum to occlude the hole. The advantage of the catheterization is that the treatment can be performed by making a tiny skin incision (only a few millimeters) at the base of the leg (inguinal region) which is an inconspicuous body area without performing a thoracotomy requiring general anesthesia.

Japanese Unexamined Patent Application Publication No. 2008-512139 (Patent Literature 1) discloses an assembly (occluder) used for catheterization for the atrial septal defect. This assembly seals a passageway (defect hole) in the heart. The assembly includes: a closure device for hermetically sealing the passageway of the heart that includes a first anchor adapted to be placed proximate a first end of the passageway, a second anchor adapted to be placed proximate a second end of the passageway, and a flexible extension material adapted to extend through the passageway and be connected to the first and second anchors, the second anchor being movable relative to the flexible extension material to change a length of the flexible extension material between the first and second anchors; and a supply system for delivering the closure device to the passageway of the heart, a supply device being designed to move within a lumen of a guide catheter and including a wire for controlling movement of the second anchor along the flexible extension material.

Patent Literature 1 discloses that a patent foramen oval (PFO) closure device (occluder) includes a left atrial anchor, a right atrial anchor, a tether, and a lock, and the left atrial anchor, the right atrial anchor coupled to the left atrial anchor via the tether, and the lock remain in the heart to seal the PFO.

### CITATION LIST

### Patent Literature

Patent Literature 1: Japanese Unexamined Patent Application Publication No. 2008-512139

### SUMMARY OF INVENTION

### Technical Problem

The patch operation involves usage of the cardiopulmonary bypass and poses high invasiveness, resulting in the problem of a long hospitalization period. In contrast, the catheterization is preferable because it does not use the cardiopulmonary bypass and poses low invasiveness, which is conducive to reduction in the hospitalization period.

As disclosed in Patent literature 1, the left atrium anchor and the right atrium anchor remain in the heart. Each of the left atrium anchor and the right atrium anchor includes equal to or more than one arm, and the arm(s) extend(s) radially outwardly from a hub. The arm is preferably formed by a rolled sheet made of a nickel-titanium two-component alloy. A defect hole is occluded by expanding the left atrium anchor and the right atrium anchor in a living body. In this case, the expansion of the anchors is once started, it is difficult to cause the anchors to recover their original states. As disclosed in Patent literature 1, the anchors have to be folded by means of a dedicated retrievable device which has a complicated structure and is difficult to operate from outside the living body.

However, for example, in the event that the anchor is accidentally caught in a biological tissue within the atrium to damage the biological tissue, there may be no time enough to fold the anchor by such a dedicated retrievable device. In this case, there is no other choice but to start the thoracotomy immediately. This finally results in the problem of undergoing the highly invasive thoracotomy.

Further, the defect hole occluder made of metal remains in the body through the whole life, and there is therefore the problem of fear of long-term failure.

The present invention has been made in view of the above-mentioned problems in the conventional art, and an object thereof is to provide a medical material that is capable of being released and placed at a treatment site in a living body, enables low invasive catheterization with easy operations without a complicated structure, and has almost no fear of long-term failure even when remaining in the body.

### Solution to Problem

In order to accomplish the above object, the medical material according to an aspect of the present invention takes the following technical means.

That is, according to an aspect of the present invention, there is provided a medical material that is formed by a cylindrical body having a stitch-like structure using a filamentary material, and the medical material has such a shape that a cylinder diameter of a substantially central part of the cylindrical body is smaller than cylinder diameters of other parts, is formed with a first cylinder part on a side of a first end part in a cylindrical body lengthwise direction of the medical material and a second cylinder part on a side of the other end part, with the substantially central part as a center, includes an elastic member both ends of which are respectively engaged with the filamentary material at the first end part and the filamentary material at the second end part, the elastic member being through inner parts of the first cylinder part and the second cylinder part from the side of the first end part to the side of the second end part side via the substantially central part, and an outer cylindrical body is provided at an outermost layer of the medical material so as to cover a whole of the cylindrical body having the stitch-like structure from an outer side, the outer cylindrical body being a cylinder-shaped body obtained by knitting a filamentary material same as or different from the filamentary material, and both ends of the outer cylindrical body being respectively joined to both ends of the cylindrical body.

It is preferable that the outer cylindrical body can be joined to the cylindrical body at at least one place in addition to the both ends of the outer cylindrical body.

It is further preferable that when the elastic member is in a compressed state, the first end part and the second end part can come close to each other with the substantially central part as the center, and the cylinder diameters of the other parts can be increased.

It is still further preferable that when the elastic member is in a compressed state, the cylinder diameters of the other parts can be increased to sizes corresponding to a defect hole to be closed by the medical material.

It is still further preferable that when the elastic member is in a stretched state, the first end part and the second end part can be separated from each other with the substantially central part as the center, and the cylinder diameters of the other parts can be decreased.

It is still further preferable that when the elastic member is in a stretched state, the cylinder diameters of the other parts can be decreased to sizes corresponding to a catheter in which the medical material is contained.

It is still further preferable that the elastic member can be formed by a coil spring having a smaller diameter than the cylinder diameter of the substantially central part.

It is still further preferable that the shape can be a sandglass shape, figure-of-eight shape, or double spindle shape.

It is still further preferable that end parts of the elastic member can be joined to small cylinder parts provided outside the cylindrical body having the stitch-like structure and capable of being engaged with an operation wire.

It is still further preferable that the filamentary material forming the cylindrical body having the stitch-like structure or the filamentary material forming the outer cylindrical body can be made of a biological absorption material.

It is still further preferable that a porous cylindrical layer formed by any one of a nonwoven fabric, sponge, film, and composite body of them made of a bioabsorbable material can be arranged on an inner surface of the cylindrical body.

### Advantageous Effects of Invention

The medical material according to the present invention is capable of being released and placed at a treatment site in a living body and enables low invasive catheterization with easy operations without a complicated structure. Furthermore, the medical material according to the present invention has almost no fear of long-term failure even when remaining in the body.

### BRIEF DESCRIPTION OF DRAWINGS

FIG. 1 is an overall view of a defect hole closing material 100 as an example of a medical material according to the present invention (when a coil spring 140 is in a compressed state).

FIG. 2A is an overall view when the coil spring 140 is in an intermediate state in the defect hole closing material 100 as the example of the medical material according to the present invention.

FIG. 2B is an overall view other than an outer cylindrical body when the coil spring 140 is in the intermediate state in the defect hole closing material 100 as the example of the medical material according to the present invention.

FIG. 2C is an overall view of only the outer cylindrical body when the coil spring 140 is in the intermediate state in the defect hole closing material 100 as the example of the medical material according to the present invention.

FIG. 2D is an overall view in the case where joint parts are provided only at both end parts when the coil spring 140 is in the intermediate state in the defect hole closing material 100 as the example of the medical material according to the present invention.

FIG. 3 is an overall view of the defect hole closing material 100 as the example of the medical material according to the present invention (when the coil spring 140 is in a stretched state).

FIG. 4 is an overall view of the defect hole closing material 100 as the example of the medical material according to the present invention (when the coil spring 140 is in the compressed state and the stretched state).

FIG. 5A is a partial side view of the defect hole closing material 100 in FIG. 2A.

FIG. 5B is a cross-sectional view along A-A in FIG. 5A.

FIG. 6 is a conceptual view when the defect hole closing material 100 as the example of the medical material according to the present invention is used for catheterization for an atrial septal defect.

FIG. 7 is an enlarged view (part 1) of a part B in FIG. 6 illustrating the procedure of the catheterization.

FIG. 8 is an enlarged view (part 2) of the part B in FIG. 6 illustrating the procedure of the catheterization.

FIG. 9 is an enlarged view (part 3) of the part B in FIG. 6 illustrating the procedure of the catheterization.

FIG. 10 is an overall view of a defect hole closing material 400 as an example of a medical material according to a variation of the present invention (when the coil spring 140 is in a compressed state).

FIG. 11 is an overall view of the defect hole closing material 400 as the example of the medical material according to the variation of the present invention (when the coil spring 140 is in an intermediate state).

FIG. 12 is a partial enlarged view of FIG. 11.

### DESCRIPTION OF EMBODIMENTS

Hereinafter, a medical material according to the present invention will be described in detail with reference to the drawings. While the following describes a defect hole closing material for use in catheterization as an example of the medical material according to the present invention, it is suitably applicable also to closure of another opening or passageway, for example, another opening in the heart due to a ventricular septal defect, patent ductus arteriosus, or the like, and an opening or a passageway at another site of a living body (for example, stomach), due to an arteriovenous fistula or the like. Accordingly, the defect hole closing material according to an embodiment of the present invention is not limited to be used for the closure of a hole arising from the atrial septal defect.

Moreover, although in the following embodiment, a stitch-like structure of a defect hole closing material (occluder) 100 will be described as an object obtained by knitting a bioabsorbable fiber (an example of a filamentary material), the present invention is not limited thereto. It is sufficient that the defect hole closing material enables catheterization adapted to close a defect hole formed in a living body, and therefore, its stitch-like structure may be knitted with a filamentary material other than the bioabsorbable fiber so long as the material has a first characteristic to a fourth characteristic as will be described later and exhibits a first action to a fourth action. Such a filamentary material preferably has a certain degree of hardness for the sake of form retainability (shape retainability).

### [Configuration]

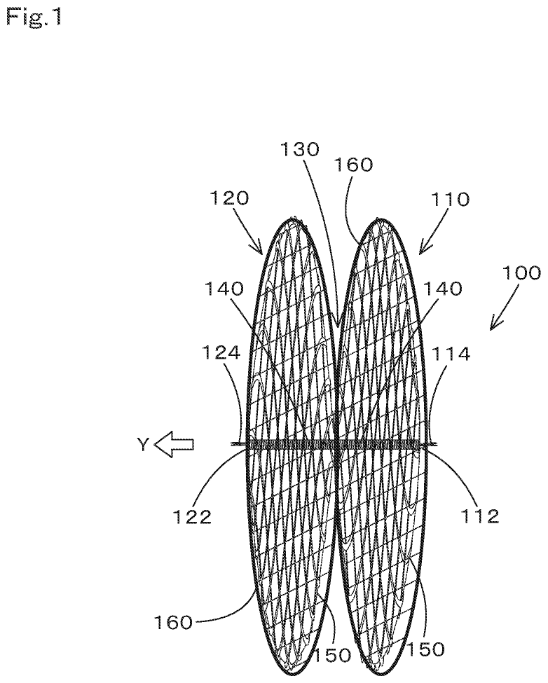

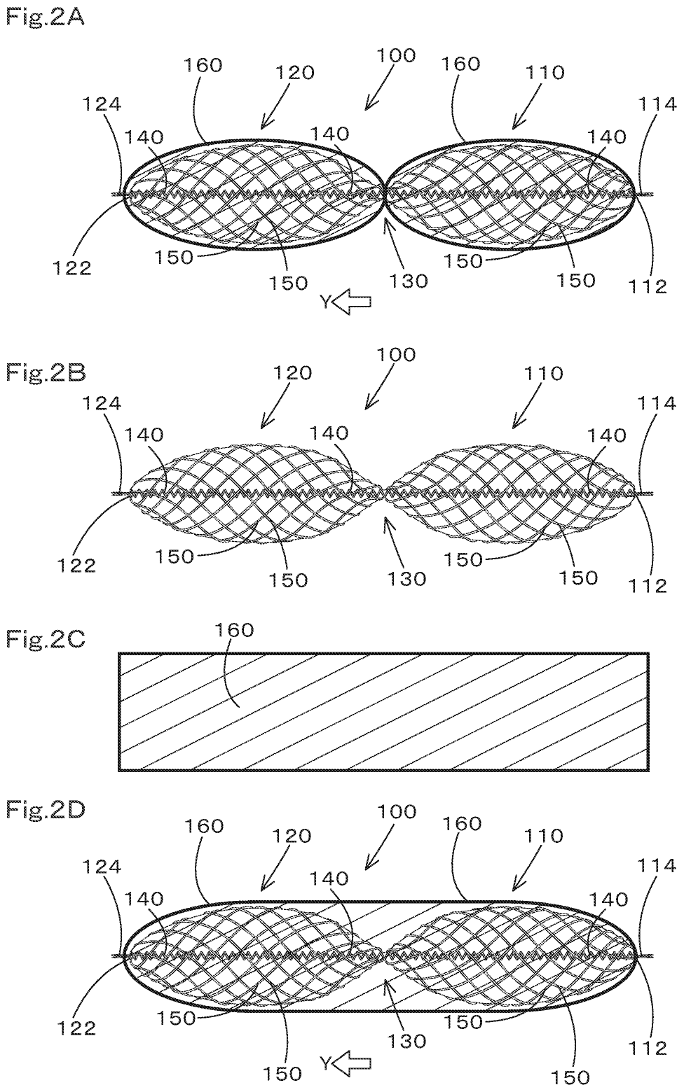

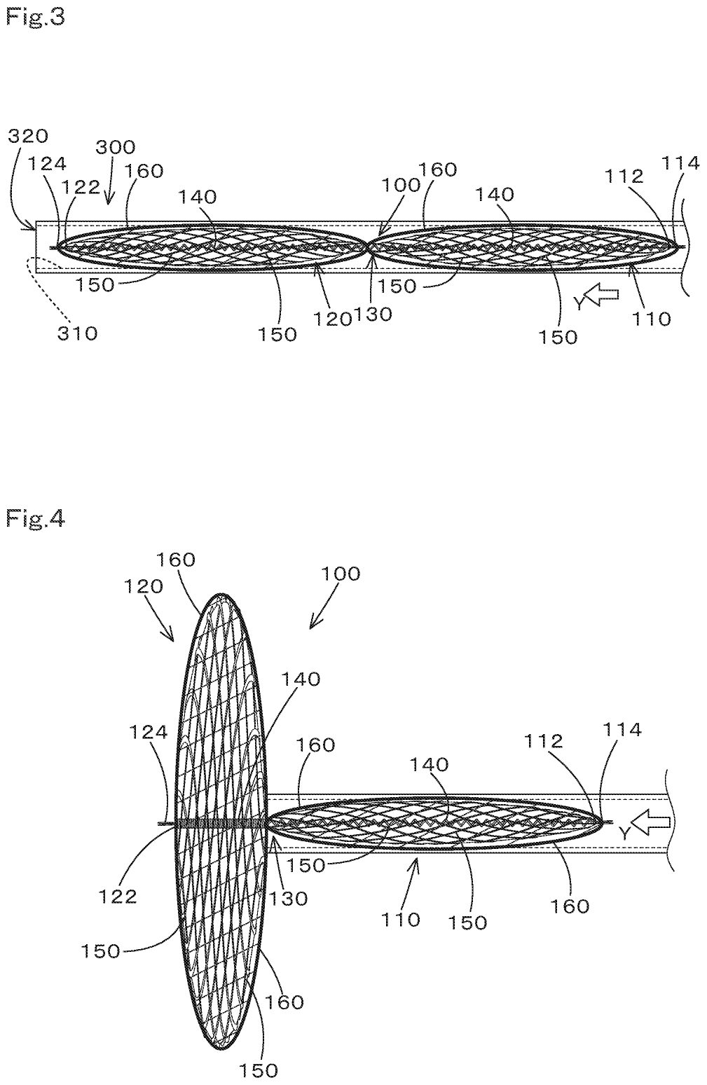

FIG. 1 is an overall view of the defect hole closing material 100 according to the embodiment (when a coil spring 140 is in a compressed state), FIGS. 2A to 2D are overall views of the defect hole closing material 100 (when the coil spring 140 is in an intermediate state), FIG. 3 is an overall view of the defect hole closing material 100 (when the coil spring 140 is in a stretched state), and FIG. 4 is an overall view of the defect hole closing material 100 (when the coil spring 140 is in the compressed state and in the stretched state). FIG. 2A is the overall view of the defect hole closing material 100, FIG. 2B is an overall view of the defect hole closing material 100 other than an outer cylindrical body 160. FIG. 2C is an overall view of only the outer cylindrical body 160. FIG. 2D is an overall view in the case where joint parts are provided only at both end parts. As will be described later, the largest characteristic point of the defect hole closing material 100 is that the outer cylindrical body 160 obtained by knitting a fiber (having bioabsorbability) into a cylindrical shape is put as an outermost layer of the defect hole closing material 100, and both ends of the outer cylindrical body 160 are joined to the cylindrical body having the stitch-like structure using a filamentary material 114. As described above, the outer cylindrical body 160 is the cylindrical body obtained by knitting the fiber as a soft material, and therefore, the shape thereof is easily deformed and is not kept to be a vertically and laterally symmetrical shape all the time (is not illustrated to be an exact vertically and laterally symmetrical oval shape when viewed from the lateral surface). The outer cylindrical body 160 is however illustrated to have the exact vertically and laterally symmetrical oval shape when viewed from the lateral surface in accordance with change in the shape of the cylindrical body having the stitch-like structure using the filamentary material 114 in these drawings. FIG. 3 is a view illustrating a state where the whole of the defect hole closing material 100 is contained in a catheter 300, and FIG. 4 is a view illustrating a state where half of the defect hole closing material 100 (a side of a first cylinder part 110) is contained in the catheter 300. When pushing the defect hole closing material 100 that is wholly contained in the catheter 300 (in a space formed by an inner wall 310) illustrated in FIG. 3 from the side of the first cylinder part 110 in the direction indicated by an arrow Y to push out a second cylinder part 120 through an opening 320 of the catheter 300, the state of FIG. 4 is made. When further pushing out the first cylinder part 110 in the direction indicated by the arrow Y, the state of FIG. 1 is made. Note that the states of the defect hole closing material 100 illustrated in FIGS. 2A to 2D are virtual states where the coil spring 140 is in the intermediate state between the compressed state and the stretched state.

As illustrated in these drawings, the defect hole closing material 100 is roughly formed by a cylindrical body having the stitch-like structure using the filamentary material and has such shape that the cylindrical diameter of a substantially central part 130 of the cylindrical body is smaller than the cylindrical diameters of the other parts, and the first cylinder part 110 on a side of a first end part 112 in the cylindrical body lengthwise direction of the defect hole closing material 100 and the second cylinder part 120 on a side of the other end part (a second end part 122) are formed with the substantially central part 130 as a center. A characteristic point is to include, as an example of an elastic member, the coil spring 140 both ends of which are respectively engaged with the filamentary material 124 at the first end part 112 and the filamentary material 124 at the second end part 122 and that is inserted through inner parts of the first cylinder part 110 and the second cylinder part 120 from the side of the first end part 112 to the side of the second end part 122 via the substantially central part 130. Even in the case other than the coil spring 140, the elastic member is only required to be a member having elasticity and capable of exhibiting actions as will be described later with the elasticity and is not limited to the coil spring 140.

Another characteristic point is in that the outer cylindrical body 160 is provided at the outermost layer of the defect hole closing material 100 so as to cover a whole of the cylindrical body having the stitch-like structure using the filamentary material from an outer side. Furthermore, the outer cylindrical body 160 is a cylinder-shaped body obtained by knitting a filamentary material that is the same as or differs from the filamentary material forming the cylindrical body having the stitch-like structure. Moreover, the both ends of the outer cylindrical body 160 are respectively joined to both ends of the cylindrical body having the stitch-like structure. To be more specific, as illustrated in FIG. 2D, the filamentary material 114 at the first end part 112 and an end part of the outer cylindrical body 160 are joined to each other, and the filamentary material 124 at the second end part 122 and an opposite-side end part of the outer cylindrical body 160 are joined to each other.

It is further preferable that the outer cylindrical body 160 be joined to the cylindrical body having the stitch-like structure at at least one place (in this example, one place in the vicinity of the substantially central part 130) in addition to the both ends of the outer cylindrical body 160. To be more specific, as illustrated in FIG. 2A and the like, the filamentary material 114 at the first end part 112 and the end part of the outer cylindrical body 160 are joined to each other, the filamentary material 124 at the second end part 122 and the opposite-side end part of the outer cylindrical body 160 are joined to each other, and in addition thereto, the filamentary material (specifically, a bioabsorbable fiber 150) at the substantially central part 130 and a substantially central part of the outer cylindrical body 160 are joined to each other. As the joint position between the outer cylindrical body 160 and the cylindrical body having the stitch-like structure, it should not be limited to at least one place in addition to the both ends, and one place in the vicinity of the substantially central part 130 is merely an example. The joint position is preferably at least one place but may be equal to or more than two places (equal to or more than four places when the both ends are included).

Although a joining method is not limited, they are preferably joined to each other by interweaving the filamentary materials thereof (tying them using another bioabsorbable fiber if necessary) because the cylindrical body having the stitch-like structure is made of the filamentary material (to be more specific, the bioabsorbable fiber 150) and the outer cylindrical body 160 is also made of the filamentary material (that may be the same as or differ from the bioabsorbable fiber 150 as the filamentary material forming the cylindrical body having the stitch-like structure) (both of them are made of the filamentary materials).

It is preferable that the filamentary material forming the cylindrical body having the stitch-like structure be the bioabsorbable fiber 150 and the filamentary material forming the outer cylindrical body 160 be a biological absorption material.

The outer cylindrical body 160 needs to have flexibility that allows the shape of the outer cylindrical body 160 to change along the outer surface shape of the cylindrical bodies (the first cylinder part 110 and the second cylinder part 120) as the other parts than the substantially central part 130 in the defect hole closing material 100 with increase/decrease in the cylinder diameters of the cylindrical body. The outer cylindrical body 160 has such flexibility because it is the cylindrical body obtained by knitting the filamentary material that is the same as or differs from the filamentary material (in this example, the bioabsorbable fiber 150) forming the cylinder-shaped body having the stitch-like structure (because of flexibility of the fiber).

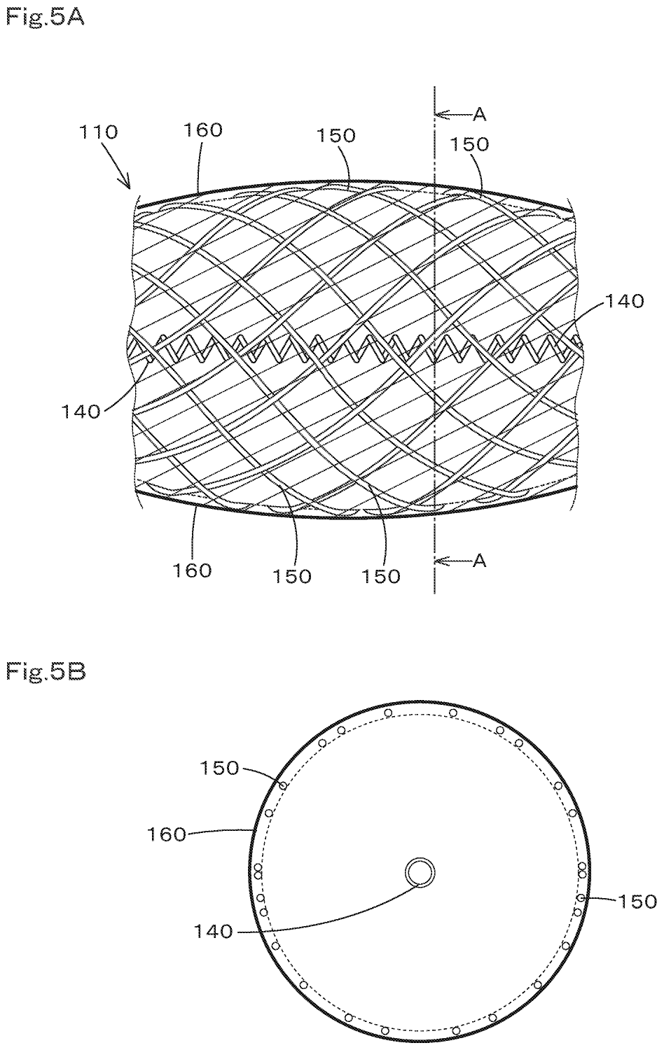

FIG. 5A is a partial side view of the defect hole closing material 100 and FIG. 5B is a cross-sectional view along A-A in FIG. 5A. Note that although FIG. 5B is the cross-sectional view of the defect hole closing material 100 (to be more specific, of the first cylinder part 110), FIG. 5B illustrates only the cross-sections of the coil spring 140, the bioabsorbable fiber 150, and the outer cylindrical body 160 (although the cross-section of the filamentary material is observed in the cross-section of the outer cylindrical body 160, the cross-section of the outer cylindrical body 160 is indicated by a circular curve in order to distinguish it) and does not illustrate stitches of the bioabsorbable fiber 150 viewable from a direction indicated by an arrow A. In addition, in FIG. 1 to FIGS. 5A and 5B, in order to facilitate the understanding of the presence of the coil spring 140 and the stitches of the bioabsorbable fiber 150, the bioabsorbable fiber 150 arranged on the deep side of the page is not illustrated, and in order to facilitate the understanding of an appearance shape of the defect hole closing material 100, the appearance shape of the defect hole closing material 100 is indicated by dotted line in some portions.

In these drawings (particularly in FIG. 2B illustrating the defect hole closing material 100 including no outer cylindrical body 160), the defect hole closing material 100 is formed by the two cylindrical bodies (the first cylinder part 110 and the second cylinder part 120) made of the bioabsorbable material and having the stitch-like structures and has a shape formed by such two cylindrical bodies, which is called, for example, a sandglass shape, a figure-of-eight shape, a double spindle shape (the shape of two continuous elongated rod-like spindle-shaped objects whose middles are thick and both ends are thin), or a peanut shape (an appearance shape of a peanut shell containing two nuts). The defect hole closing material 100 having the above-mentioned shape has such a shape that the substantially central part 130 is narrowed so as to make the cylinder diameter of the substantially central part 130 of the cylindrical body smaller than the cylinder diameters of the other parts thereof. That is, the first cylinder part 110 on the first end part 112 side and the second cylinder part 120 on the second end part 122 side are formed with the substantially central part 130 as the center.

For the defect hole closing material 100 including no outer cylindrical body 160 illustrated in FIG. 2B, the outer cylindrical body 160 illustrated in FIG. 2C is separately prepared (prepared by knitting, into the cylindrical shape, the filamentary material that is the same as or differs from the filamentary material (the bioabsorbable fiber 150) forming the cylindrical body having the stitch-like structure). Then, the outer cylindrical body 160 illustrated in FIG. 2C is put as the outermost layer of the defect hole closing material 100 including no outer cylindrical body 160 illustrated in FIG. 2B. Subsequently, as illustrated in FIG. 2D, the filamentary material 114 at the first end part 112 and the end part of the outer cylindrical body 160 are joined to each other and the filamentary material 124 at the second end part 122 and the opposite-side end part of the outer cylindrical body 160 are joined to each other, so that the outer cylindrical body 160 is fixed to the cylindrical body having the stitch-like structure. Alternatively, as illustrated in FIG. 2A and others, the filamentary material 114 at the first end part 112 and the end part of the outer cylindrical body 160 are joined to each other, the filamentary material 124 at the second end part 122 and the opposite-side end part of the outer cylindrical body 160 are joined to each other, and in addition, the filamentary material (to be more specific, the bioabsorbable fiber 150) at the substantially central part 130 and the substantially central part of the outer cylindrical body 160 are joined to each other, so that the outer cylindrical body 160 is fixed to the cylindrical body having the stitch-like structure.

After the outer cylindrical body 160 is fixed to the cylindrical body having the stitch-like structure, the outer cylindrical body 160 can be easily deformed in accordance with change in the shape of the cylindrical body (the first cylinder part 110, the second cylinder part 120, and the central part 130) having the stitch-like structure because the outer cylindrical body 160 is the cylinder-shaped body obtained by knitting the fiber as the soft material and the shape thereof is easily deformed with the following configuration. That is, the outer cylindrical body 160 is the cylindrical body obtained by knitting the filamentary material that is the same as or differs from the filamentary material (the bioabsorbable fiber 150) forming the cylindrical body having the stitch-like structure, and at least the filamentary material 114 at the first end part 112 and the end part of the outer cylindrical body 160 are joined to each other and the filamentary material 124 at the second end part 122 and the opposite-side end part of the outer cylindrical body 160 are joined to each other (in addition thereto, the outer cylindrical body 160 is joined at the substantially central part 130). The outer cylindrical body 160 joined to the cylindrical body having the stitch-like structure to be integrated as the defect hole closing material 100 is thus deformed in a similar manner together with the cylindrical body having the stitch-like structure. Therefore, in the following, change in the shape of the outer cylindrical body 160 is represented by the change in the shape of the cylindrical body having the stitch-like structure, and the change in the shape of the outer cylindrical body 160 itself is not described in some cases.

Although not limited, in the defect hole closing material 100, the first cylinder part 110 and the second cylinder part 120 are integrally knitted such that the cylinder diameter of the substantially central part 130 is made smaller than the cylinder diameters of the other parts, and the defect hole closing material 100 is formed into, as the whole shape, the sandglass shape, figure-of-eight shape, double spindle shape, or peanut shape formed by the two cylindrical bodies.

In this case, the whole shape of the defect hole closing material 100 is formed by using a frame (a three-dimensional paper pattern) of such a sandglass shape, figure-of-eight shape, double spindle shape, or peanut shape to knit one bioabsorbable fiber 150 in conformity with the frame. Further, although not limited, the defect hole closing material 100 may be formed into the sandglass shape, figure-of-eight shape, double spindle shape, or peanut shape formed by the two cylindrical bodies as the whole shape of the defect hole closing material 100 in the following manner. That is, the first cylinder part 110 and the second cylinder part 120 are integrally knitted to knit a cylindrical body having a substantially uniform diameter, and then, the substantially central part 130 is tied and/or is thermally set and so on to thereby make the cylinder diameter of the substantially central part 130 be smaller than the cylinder diameters of the other parts. Thereafter, the tying of the substantially central part 130 and/or the thermal setting thereof is/are released to form the substantially central part 130 having the cylinder diameter that is larger than the diameter of the coil spring 140. As will be described in detail later, the above-mentioned shape makes it possible to achieve the following change in shape. That is, when pushing the defect hole closing material 100 that is wholly contained in the catheter 300 (in the space formed by the inner wall 310) illustrated in FIG. 3 from the first cylinder part 110 side in the direction indicated by the arrow Y to push out the second cylinder part 120 through the opening 320 of the catheter 300, the second cylinder part 120 is released from the space formed by the inner wall 310 of the catheter 300 to compress the coil spring 140 in the second cylinder part 120, and the state of FIG. 4 is made. When further pushing out the first cylinder part 110 in the direction indicated by the arrow Y, the first cylinder part 110 is released from the space formed by the inner wall 310 of the catheter 300 to compress the coil spring 140 in the first cylinder part 110, and the state of FIG. 1 is made. In this case, the outer cylindrical body 160 is deformed in accordance with the change in shapes of the second cylinder part 120 and the first cylinder part 110. The same holds true for the following, and the change in the shape of the outer cylindrical body 160 is not described in some cases.

Furthermore, the defect hole closing material 100 includes the coil spring 140 one end of which is engaged with the first end part 112 (for example, is hooked on a loop of the filamentary material 114 at the first end part 112), the other end of which is engaged with the second end part 122 (for example, is hooked on a loop of the filamentary material 124 at the second end part 122), and that is inserted through the inner parts of the first cylinder part 110 and the second cylinder part 120 from the first end part 112 side to the second end part 122 side via the substantially central part 130. The loop-shaped filamentary material 114 and filamentary material 124 are formed by the bioabsorbable fiber 150.

As illustrated in FIG. 1, when the coil spring 140 is in the compressed state, the first end part 112 and the second end part come close to each other with the substantially central part 130 as the center, and the cylinder diameters of the first cylinder part 110 and the second cylinder part 120 as the other parts than the substantially central part 130 are increased together with the outer cylindrical body 160. Particularly preferably, when the coil spring 140 is in the compressed state, the cylinder diameters of the first cylinder part 110 and the second cylinder part 120 as the other parts than the substantially central part 130 are increased together with the outer cylindrical body 160 to sizes corresponding to a defect hole to be closed by the defect hole closing material 100.

As illustrated in FIG. 3, when the coil spring 140 is brought into the stretched state by containing the defect hole closing material 100 in the catheter 300 and so on, the first end part 112 and the second end part 122 are separated from each other with the substantially central part 130 as the center, and the cylinder diameters of the first cylinder part 110 and the second cylinder part 120 as the other parts are decreased together with the outer cylindrical body 160. Particularly preferably, when the coil spring 140 is in the stretched state, the cylinder diameters of the first cylinder part 110 and the second cylinder part 120 as the other parts are decreased together with the outer cylindrical body 160 to sizes corresponding to the catheter 300 in which the defect hole closing material 100 is to be contained.

As described above, by using the coil spring 140 having the diameter smaller than the cylinder diameter of the substantially central part 130, the first end part 112 and the second end part 122 as the other end part in the lengthwise direction of the cylindrical body in the defect hole closing material 100 can be brought close to or separated from each other. When the coil spring 140 is made into the compressed state, as illustrated in FIG. 1, the first end part 112 and the second end part 122 come close to each other to increase the cylinder diameters of the other parts than the substantially central part 130 (the cylinder diameters of body parts of the first cylinder part 110 and the second cylinder part 120) together with the outer cylindrical body 160. When the coil spring 140 is made into the stretched state, as illustrated in FIG. 3, the first end part 112 and the second end part 122 are separated from each other to decrease the cylinder diameters of the other parts than the substantially central part 130 (the cylinder diameters of the body parts of the first cylinder part 110 and the second cylinder part 120) together with the outer cylindrical body 160. Further, as illustrated in FIG. 4, when pushing the second cylinder part 120 out of the catheter 300 in the direction indicated by the arrow Y, the second cylinder part 120 the shape of which has been restricted by the inner wall 310 of the catheter 300 can freely change its shape, and the coil spring 140 causes to increase only the cylinder diameter of the body part of the second cylinder part 120 together with the outer cylindrical body 160 corresponding to the part. When further pushing the first cylinder part 110 out of the catheter 300 in the direction indicated by the arrow Y, the first cylinder part 110 the shape of which has been restricted by the inner wall 310 of the catheter 300 can also freely change its shape, and the part of the coil spring 140 that is contained in the first cylinder part 110 is also compressed to increase the cylinder diameter of the body part of the first cylinder part 110 as well together with the outer cylindrical body 160 corresponding to the part, as illustrated in FIG. 1.

As described above, basically, all of the first cylinder part 110, the second cylinder part 120, and the outer cylindrical body 160 except for the coil spring 140 are made of the bioabsorbable material. The whole of the defect hole closing material 100 excluding the coil spring 140 therefore has bioabsorbability. In addition, the change in the shape of the defect hole closing material 100 allows the treatment for closing the defect hole to be performed, and the defect hole closing material 100 including the outer cylindrical body 160 is formed while employing a material, stitch shape, fiber structure, and fiber cross section that prevent an in vivo tissue from being damaged even when the shape of the defect hole closing material 100 is thus changed in the living body.

Normally, the coil spring 140 is made of, for example, the nickel-titanium alloy and does not have the bioabsorbability. Alternatively, it may be made of a magnesium-based alloy as will be described later to have the bioabsorbability. Usage of an alloy having a property that does not transmit X rays for the coil spring 140 is advantageous in terms of reacting to X-ray imaging, and usage of the alloy having the bioabsorbability is advantageous in terms of preventing the problem of fear of long-term failure because no metallic member remains in the body through the whole life.

The bioabsorbable fiber 150 forming the first cylinder part 110 and the second cylinder part 120 is, for example, of at least one type selected from polyglycolic acid, polylactide (D, L, and DL isomers), polycaprolactone, glycolic acid-lactide (D, L, and DL isomers) copolymers, a glycolic acid-ε-caprolactone copolymer, lactide (D, L, and DL isomers)-ε-caprolactone copolymers, poly (p-dioxanone), glycolic acid-lactide (D, L, and DL isomers)-ε-caprolactone copolymers, and the like. While the selected material is used after being processed into any one of a monofilament yarn form, multifilament yarn form, twisted yarn form, braid form, and the like, the material is preferably used in the monofilament yarn form.

Further, the material of the bioabsorbable fiber 150 may be a biodegradable alloy. Examples of such a biodegradable alloy include the magnesium-based alloy as a raw material.

The bioabsorbable fiber 150 is designed to have a diameter in a range of about 0.001 mm to 1.5 mm, and a fiber diameter and type suitable for catheterization to be applied are selected. Also, the cross section of the bioabsorbable fiber 150 may be any one of a circle, ellipse, and other different shapes (such as a star shape) under the condition that the in vivo tissue is not damaged. Further, the surface of the bioabsorbable fiber 150 may be subjected to hydrophilic treatment by plasma discharge, electron beam treatment, corona discharge, ultraviolet irradiation, ozone treatment, or the like. Moreover, the bioabsorbable fiber 150 may be subjected to application or impregnation processing with an X-ray non-permeable material (such as barium sulfide, a gold chip, and a platinum chip), adhesion processing of a medical agent (such as a medical agent suitable for catheterization for an atrial septal defect), or coating processing with a natural polymer such as collagen and gelatin or a synthetic polymer such as polyvinyl alcohol and polyethylene glycol.

The first cylinder part 110 and the second cylinder part 120 are formed as follows. That is, the bioabsorbable fiber 150 is knitted into a braid-like textile using a braiding machine with multiple (for example, 8 or 12) yarn feeders around a silicone-made rubber tube (not illustrated) having an outer diameter desired as the monofilament yarn, for example, or knitted into a cylindrical stitch-like structure having a substantially uniform diameter using a circular knitting machine (not illustrated). After the knitting, as described above, the braid-like textile or the cylindrical stitch-like structure is narrowed in the substantially central part 130 with a string made of the same material as that of the first cylinder part 110 and the second cylinder part 120 and is formed into the sandglass shape, figure-of-eight shape, double spindle shape, or peanut shape formed by the two cylindrical bodies. The cylinder diameters of the first cylinder part 110 and the second cylinder part 120 are set to be smaller than the inner diameter of the catheter in the diameter-decreased states and to those preferable for the catheterization for the atrial septal defect in the diameter-increased states. For example, the cylinder diameters of the first cylinder part 110 and the second cylinder part 120 in the diameter-increased states are in a range of 5 mm to 80 mm, and preferably in a range of about 15 mm to 25 mm. In addition, the lengths of the first cylinder part 110 and the second cylinder part 120 and the density of the stitch-like structure of the defect hole closing material 100 are also set to be sizes preferable for the catheterization for the atrial septal defect. Note that the cylinder diameters and lengths of the first cylinder part 110 and the second cylinder part 120 do not have to be the same but may be changed so as to be preferable for the catheterization for the atrial septal defect.

The bioabsorbable material forming the outer cylindrical body 160 is not particularly limited, and examples thereof include synthetic absorbable polymers such as polyglycolic acid, polylactide (D, L, and DL isomers), polycaprolactone, glycolic acid-lactide (D, L, and DL isomers) copolymers, a glycolic acid-ε-caprolactone copolymer, lactide (D, L, and DL isomers)-ε-caprolactone copolymers, poly (p-dioxanone), and glycolic acid-lactide (D, L, and DL isomers)-ε-caprolactone copolymers. These may be used individually, or equal to or more than two types thereof may be used in combination. Among them, at least one type selected from a group consisting of polyglycolic acid, lactide (D, L, and DL isomers)-ε-caprolactone copolymers, a glycolic acid-ε-caprolactone copolymer, and glycolic acid-lactide (D, L, and DL isomers)-ε-caprolactone copolymers is preferable because they exhibit appropriate degradation behaviors, and the porous layers 160 are formed by any one of the non-woven fabric, sponge, film, and composite body of them. In particular, as a preferred mode, the non-woven fabric can be exemplified.

Further, the material of the outer cylindrical body 160 may be a biodegradable alloy. Examples of such a biodegradable alloy include the alloy based on magnesium as a raw material.

As described above, the defect hole closing material 100 according to the embodiment has the following characteristics.

(First Characteristic) The defect hole closing material 100 is formed into the sandglass shape, figure-of-eight shape, double spindle shape, or peanut shape narrowed in the substantially central part 130 and formed by the first cylinder part 110 and the second cylinder part 120.
(Second Characteristic) The defect hole closing material 100 includes the coil spring 140 one end of which is engaged with the first end part 112 (caught on the looped filamentary material 114 at the first end part 112), the other end of which is engaged with the second end part 122 (caught on the looped filamentary material 124 at the second end 122), and that is inserted through the inner parts of the first cylinder part 110 and the second cylinder part 120 from the first end part 112 side to the second end part 122 side via the substantially central part 130.
(Third Characteristic) The defect hole closing material 100 is formed by the first cylinder part 110, the second cylinder part 120, the coil spring 140 (when made of the magnesium-based alloy), and the outer cylindrical body 160, and these components are all made of the bioabsorbable material (the coil spring 140 does not necessarily have the bioabsorbability).
(Fourth characteristic) The outer cylindrical body 160 obtained by knitting the fiber (having bioabsorbability as an example) into the cylindrical shape is put as the outermost layer of the defect hole closing material 100, and the both ends of the outer cylindrical body 160 are joined to the cylindrical body having the stitch-like structure using the filamentary material 114 for integration.

With the first characteristic and the second characteristic, in the defect hole closing material 100 contained in the catheter 300, when pushing the second cylinder part 120 out of the catheter 300, the second cylinder part 120 the shape of which has been restricted by the inner wall 310 of the catheter 300 can freely change its shape together with the outer cylindrical body 160, and only the part of the whole of the coil spring 140 that is contained in the second cylinder part 120 can be compressed to increase only the cylinder diameter of the body part of the second cylinder part 120 together with the outer cylindrical body 160, and when further pushing the first cylinder part 110 out of the catheter 300, the first cylinder part 110 the shape of which has been restricted by the inner wall 310 of the catheter 300 can also freely change its shape together with the outer cylindrical body 160, and the part of the whole of the coil spring 140 that is contained in the first cylinder part 110 can also be compressed to increase the cylinder diameter of the body part of the first cylinder part 110 as well together with the outer cylindrical body 160. The outer cylindrical body 160 expands to the size corresponding to the defect hole to be closed by the defect hole closing material 100 with the above-mentioned increase in the cylinder diameters of the body parts.

In particular, the defect hole closing material 100 is suitable for the catheterization for the atrial septal defect because it provides the following actions.

(First Action) The defect hole closing material 100 can be set in the catheter 300 by stretching the whole of the coil spring 140 to make the cylinder diameter of the defect hole closing material 100 including the outer cylindrical body 160 smaller than the inner diameter of the catheter 300.
(Second Action) The defect hole closing material 100 is set in the catheter 300 and sent to a position of a hole opening in the atrial septum. Then, when pushing the first end part 112 with an applicator or the like in a living body to push the second cylinder part 120 out of the catheter 300 into the living body, the coil spring 140 in the second cylinder part 120 is compressed to increase the cylinder diameter of the body part of the second cylinder part 120 together with the outer cylindrical body 160. When further pushing the first end part 112 with the applicator or the like to push the first cylinder part 110 out of the catheter 300 into the living body, the coil spring 140 in the first cylinder part 110 is also compressed to increase the cylinder diameter of the body part of the first cylinder part 110 as well together with the outer cylindrical body 160. The first cylinder part 110 arranged on the right atrium side and the second cylinder part 120 arranged on the left atrium side therefore come close to each other with the substantially central part 130 as the center to occlude the hole opening in the atrial septum together with the outer cylindrical body 160.
(Third Action) The components (excluding the coil spring 140 in some cases) forming the defect hole closing material 100 are all made of the bioabsorbable material, so that they are finally absorbed in the living body and fear of long-term failure is almost eliminated.
(Fourth Action) The outer cylindrical body 160 included in the defect hole closing material 100 can hold the overall shape of the defect hole closing material 100. The outer cylindrical body 160 can therefore facilitate an operation when the defect hole closing material 100 is inserted into the catheter 300 outside the living body and occlude the hole opening in the atrial septum together with the first cylinder part 110 and the second cylinder part 120 in the living body.

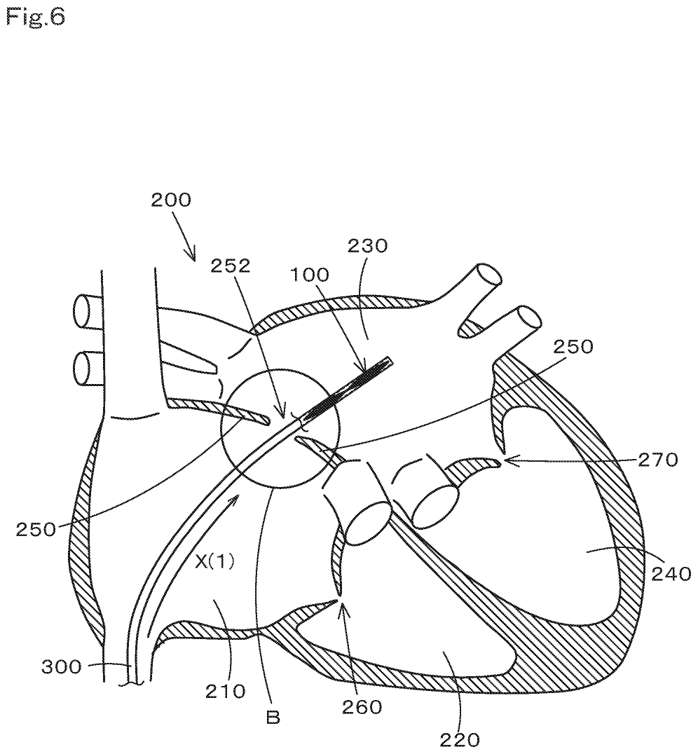

In order to facilitate understanding of the above-mentioned actions, the case where the defect hole closing material 100 is used for the catheterization for the atrial septal defect will be described with reference to FIG. 6 to FIG. 9.

### [Usage Mode]

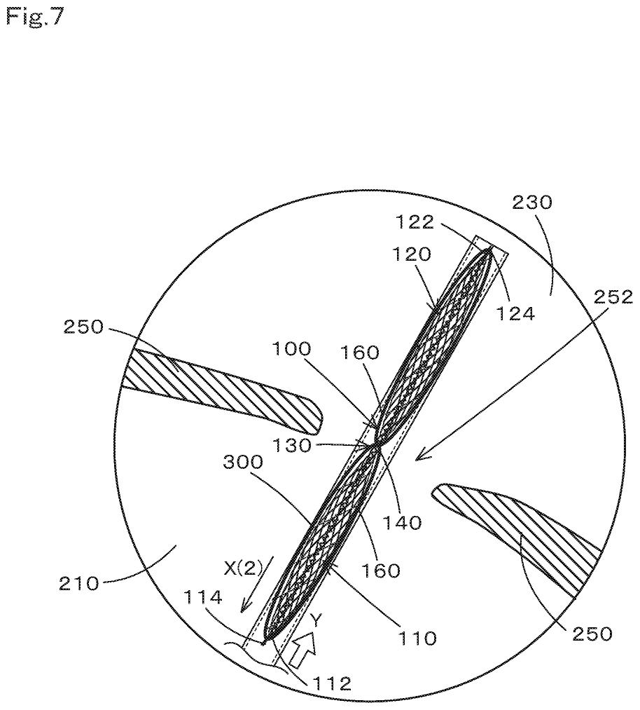

FIG. 6 is a conceptual view when the defect hole closing material 100 is used for the catheterization for the atrial septal defect, and FIG. 7 to FIG. 9 are enlarged views of a part B in FIG. 6 illustrating the procedure of the catheterization. In the following, only matters specific to the usage mode of the defect hole closing material 100 according to the embodiment are described. Since general matters are the same as those of well-known catheterization for the atrial septal defect, detailed description thereof is not repeated here.

As illustrated in FIG. 6, a heart 200 of a human includes two atria and two ventricles, i.e., the right atrium 210 connected to a superior vena cava and an inferior vena cava to receive venous blood from the whole body, a right ventricle 220 connected to the right atrium 210 via a pulmonary artery and a tricuspid valve 260 to feed venous blood to lungs, the left atrium 230 connected to a pulmonary vein to receive arterial blood from the lungs, and a left ventricle 240 connected to the left atrium 230 via an aorta and a mitral valve 270 to feed arterial blood to the whole body. The atrial septal defect is a disorder in which the defect hole 252 opens in an atrial septum 250 separating between the right atrium 210 and the left atrium 230. Note that in FIG. 6, the tip side of the catheter 300 is indicated by a virtual line and the defect hole closing material 100 contained in the catheter 300 is indicated by a solid line for easy understanding.

First, outside the living body, the first end part 112 and the second end part 122 of the defect hole closing material 100 that expands to a size appropriate for the defect hole 252 are pulled in mutually separating directions, so that the whole of the coil spring 140 is stretched to make the cylinder diameter of the defect hole closing material 100 including the outer cylindrical body 160 smaller than the inner diameter of the catheter 300. Thus, the defect hole closing material 100 is set in the catheter 300. The catheter 300 containing the defect hole closing material 100 is inserted from the femoral vein (see FIG. 3) and is moved in the direction indicated by an arrow an X(1) to pass through the defect hole 252 from the right atrium 210 side, so that the catheter 300 containing the defect hole closing material 100 is brought close to the left atrium 230 side.

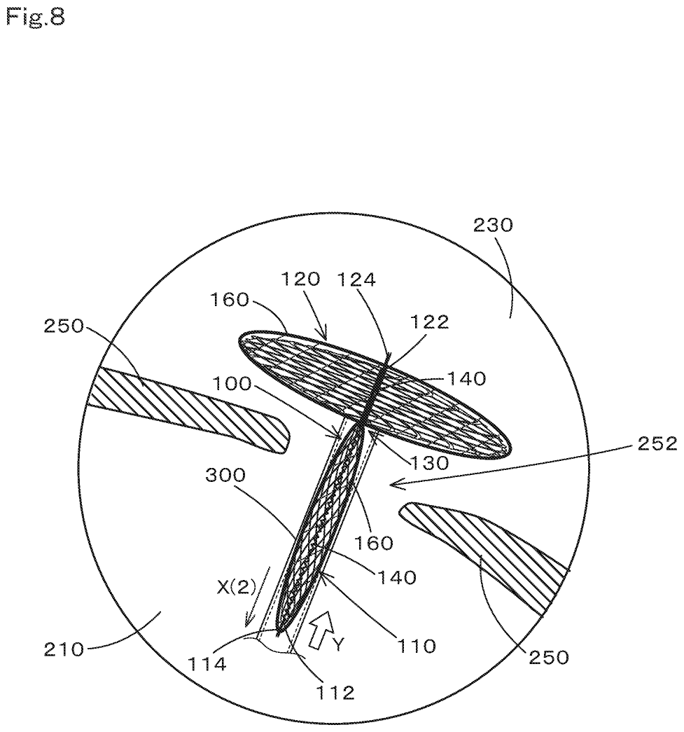

As illustrated in FIG. 6 and FIG. 7, at a position where the substantially central part 130 of the defect hole closing material 100 faces the vicinity of the defect hole 252, the catheter 300 containing the defect hole closing material 100 is stopped. In the living body, when pushing the second cylinder part 120 out of the catheter 300 with the applicator or the like in the direction indicated by the arrow Y, the second cylinder part 120 the shape of which has been restricted by the inner wall 310 of the catheter 300 can freely change its shape, and only the part of the coil spring 140 that is contained in the second cylinder part 120 is compressed to increase only the cylinder diameter of the body part of the second cylinder part 120 and the cylinder diameter of the outer cylindrical body 160 corresponding to the part as illustrated in FIG. 8.

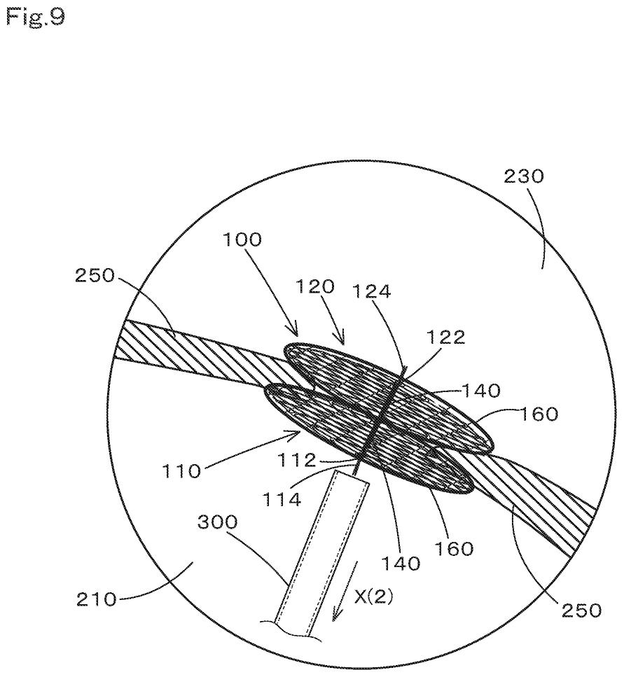

When further pushing the first cylinder part 110 out of the catheter 300 with the applicator or the like in the direction indicated by the arrow Y, the first cylinder part 110 the shape of which has been restricted by the inner wall 310 of the catheter 300 can also freely change its shape, and the part of the coil spring 140 that is contained in the first cylinder part 110 is also compressed to increase the cylinder diameter of the body part of the first cylinder part 110 and the cylinder diameter of the outer cylindrical body 160 corresponding to the part as well, as illustrated in FIG. 9.

That is, when pushing the defect hole closing material 100 out of the catheter 300 with the applicator or the like, the second cylinder part 120 arranged on the left atrium side and the outer cylindrical body 160 corresponding to the part first expand, and then, the first cylinder part 110 arranged on the right atrium side and the outer cylindrical body 160 corresponding to the part expand. As a result, the first cylinder part 110 arranged on the right atrium 210 side and the outer cylindrical body 160 corresponding to the part and the second cylinder part 120 arranged on the left atrium 230 side and the outer cylindrical body 160 corresponding to the part come close to each other with the substantially central part 130 (the defect hole 252) as the center, and also the first cylinder part 110 and the outer cylindrical body 160 corresponding to the part and the second cylinder part 120 and the outer cylindrical body 160 corresponding to the part expand. Finally, as illustrated in FIG. 9, the first cylinder part 110 and the outer cylindrical body 160 corresponding to the part and the second cylinder part 120 and the outer cylindrical body 160 corresponding to the part sandwich the atrial septum 250 from both side thereof, and the defect hole 252 opening in the atrial septum 250 can be occluded by the defect hole closing material 100.

After that, the catheter 300 is moved in the direction indicated by an arrow X(2) to take the catheter 300 out of the living body, thereby completing the treatment. With this treatment, in the living body (to be accurate, near the defect hole 252), the defect hole closing material 100 that is wholly made of the bioabsorbable material (the coil spring 140 is excluded in some cases) is placed. As described above, since all of the materials of the defect hole closing material 100 placed in the living body are the bioabsorbable material (the coil spring 140 is excluded in some cases) and finally absorbed in the living body, there is almost no fear of long-term failure.

In addition, when the coil spring 140 is not provided, it is necessary to fix the form of the defect hole closing material 100 to the form illustrated in FIG. 9 before the defect hole closing material 100 is placed in the living body. As a conceivable method, for example, heat fusibility is imparted to the bioabsorbable fiber 150, and the bioabsorbable fiber 150 is thermally set within the living body. The defect hole closing material 100 is however advantageous because the coil spring 140 enables the form of the defect hole closing material 100 to be fixed to the form illustrated in FIG. 9.

As described above, since the defect hole closing material 100 according to the embodiment is wholly made of the bioabsorbable material (the coil spring 140 is excluded in some cases) and is finally absorbed in the living body, there is almost no fear of long-term failure. Inclusion of the coil spring 140 enables the cylinder diameter of the defect hole closing material 100 to be easily changed together with the outer cylindrical body 160 located as the outer layer thereof. The defect hole closing material 100 can thereby be easily set in the catheter by changing the cylinder diameter of the defect hole closing material 100 and the cylinder diameter of the outer cylindrical body 160 to be decreased. In this case, the outer cylindrical body 160 included in the defect hole closing material 100 can hold the whole shape of the defect hole closing material 100, thereby facilitating the operation when the defect hole closing material 100 is inserted into the catheter 300 outside the living body. Furthermore, only by pushing the defect hole closing material 100 out of the catheter 300 at a position of the defect hole, by being provided with the coil spring 140, the cylinder diameter of the defect hole closing material 100 can be easily changed to be increased together with the outer cylindrical body 160 such that the two cylindrical bodies come close to each other, and the form can therefore be easily fixed, thereby occluding the defect hole opening in the atrial septum.

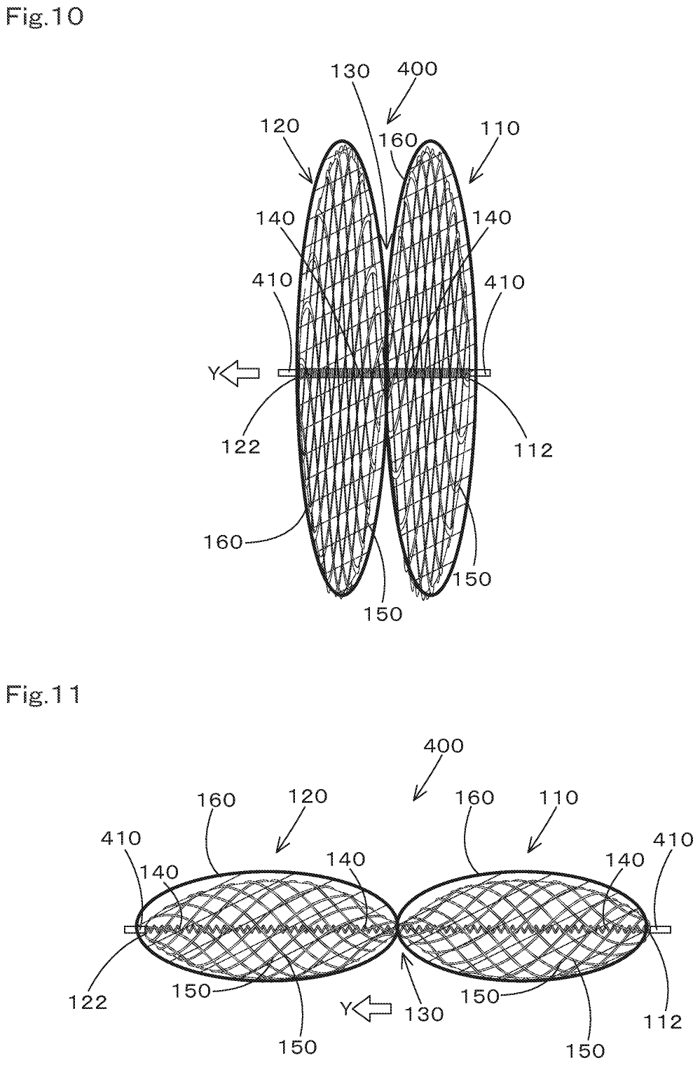

Hereinafter, a defect hole closing material (occluder) 400 as an example of a medical material according to a variation of the present invention will be described with reference to FIG. 10 to FIG. 12. The defect hole closing material 400 according to the variation is the same as the above-mentioned defect hole closing material 100 other than the following points. That is, in the defect hole closing material 400, the end parts of the elastic member (coil spring 140) in the above-mentioned defect hole closing material 100 are joined to small cylinder parts that are provided outside the cylindrical body (the first cylinder part 110 and the second cylinder part 120) having the stitch-like structure and can be engaged with an operation wire 500, and the small cylinder parts are used to join the filamentary material 114 at the first end part 112 and the end part of the outer cylindrical body 160 and join the filamentary material 124 at the first end part 122 and the opposite-side end part of the outer cylindrical body 160. Overlapped portions as those in the above description are not repeatedly described here.

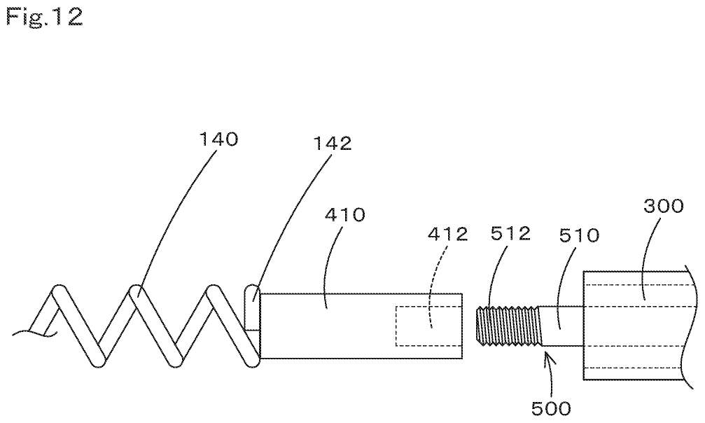

FIG. 10 is an overall view of the defect hole closing material 400 (when the coil spring 140 is in a compressed state) and corresponds to FIG. 1, FIG. 11 is an overall view of the defect hole closing material 400 (when the coil spring 140 is in an intermediate state) and corresponds to FIG. 2A to FIG. 2D, and FIG. 12 is a partial enlarged view of FIG. 11.

As illustrated in these drawings, both end parts 142 of the coil spring 140 are joined to small cylinder parts (to be more specific, cylindrical metal pieces 410) with female screw parts 412 screwable with a male screw part 512 provided on a tip part 510 of the operation wire 500 to be inserted into the catheter 300. The metal pieces 410 are provided outside the cylindrical body (the first cylinder part 110 and the second cylinder part 120) having the stitch-like structure. One end (to be more specific, the metal piece 410) of the coil spring 140 is connected to the first end part 112 (for example, a loop of the filamentary material 114 at the first end part 112 and the metal piece 410 provided at one end of the coil spring 140 are connected to each other), and the other end (to be more specific, the metal piece 410) of the coil spring 140 is connected to the second end part 122 (for example, a loop of the filamentary material 124 at the second end part 122 and the metal piece 410 provided at the other end of the coil spring 140 are connected to each other). When the coil spring 140 with the metal pieces 410 at both ends is connected as described above, the filamentary material 114 at the first end part 112 and the end part of the outer cylindrical body 160 are joined to each other, and the filamentary material 124 at the second end part 122 and the opposite-side end part of the outer cylindrical body 160 are joined to each other. The coil spring 140 having the both end parts 142 to which the metal pieces 410 are joined is inserted through the inner parts of the first cylinder part 110 and the second cylinder part 120 from the first end part 112 side to the second end part 122 side via the substantially central part 130. That is to say, the metal pieces 410 provided at both ends of the coil spring 140 are connected at the first end part 112 to which one end of the outer cylindrical body 160 is joined and the second end part 122 to which the other end of the outer cylindrical body 160 is joined, the first end part 112 and the second end part 122 being both end parts of the defect hole closing material 400. In this manner, in the defect hole closing material 400, the metal pieces 410 as the small cylinder parts are used to integrally join (or connect) the coil spring 140, the filamentary material 114 at the first end part 112, and the end part of the outer cylindrical body 160 and integrally join (or connect) the coil spring 140, the filamentary material 124 at the first end part 122, and the opposite-side end part of the outer cylindrical body 160. It should be noted that the small cylinder parts may be made of a material other than metal, and male screw parts may be provided on the metal pieces 410 whereas a female screw part may be provided on the operation wire 500.

As described above, the coil spring 140 made of the nickel-titanium alloy and the metal pieces 410 made of stainless steel can be exemplified. As a joining method in the case of the combination of such metals, joining by caulking can be exemplified.

The defect hole closing material 400 having the above-mentioned configuration is used in the same manner as the usage mode of the above-mentioned defect hole closing material 100. Particularly preferably, in the defect hole closing material 400, the both end parts 142 of the coil spring 140 and the metal pieces 410 with the female screw parts 412 screwable with the male screw part 512 provided on the tip part 510 of the operation wire 500 to be inserted into the catheter 300 are joined to each other. The defect hole closing material 400 can therefore be used in the following manner.

As illustrated in FIG. 7, outside the living body, the first end part 112 and the second end part 122 of the defect hole closing material 100 that expands to the size appropriate for the defect hole 252 are pulled in the mutually separating directions, so that the whole of the coil spring 140 is stretched to make the cylinder diameter of the defect hole closing material 100 (the cylinder diameter including the outer cylindrical body 160) smaller than the inner diameter of the catheter 300. Thus, the defect hole closing material 100 is set in the catheter 300. In this case, although not illustrated in FIG. 7, the male screw part 512 provided on the tip part 510 of the operation wire 500 to be inserted into the catheter 300 and the female screw part 412 of one of the metal pieces 410 joined to the both end parts 142 of the coil spring 140 are engaged with each other.

In the living body, as illustrated in FIG. 7 or FIG. 8, the second cylinder part 120 or the first cylinder part 110 is pushed out of the catheter 300 in the direction indicated by the arrow Y. In this case, the coil spring 140 is coupled to the tip of the operation wire 500 via the metal piece 410 (a member made of metal having no flexibility is connected to the bioabsorbable fiber 150), so that they can be pushed out in the direction indicated by the arrow Y with preferable operability by operating (pushing out) the operation wire 500 from outside the living body.

As illustrated in FIG. 9, the cylinder diameter of the body part of the second cylinder part 120 and the cylinder diameter of the body part of the first cylinder part 110 in the defect hole closing material 400 expand together with the outer cylindrical body 160. Thereafter, although not illustrated in FIG. 9, engagement between the male screw part 512 and the female screw part 412 is released by operating (rotating) the operation wire 500 from outside the living body. After that, the catheter 300 and the operation wire 500 are moved in the direction indicated by an arrow X(2) to take the catheter 300 and the operation wire 500 out of the living body. With this, treatment is completed.

As described above, the defect hole closing material 400 according to the variation can further improve the operability of the above-mentioned defect hole closing material 100.

Note that the embodiment disclosed herein should be considered to be illustrative in all respects and non-limiting. The scope of the present invention is indicated by the scope of the invention rather than the foregoing description, and all changes that come within the meaning and the range equivalent to the scope of the invention are intended to be encompassed therein.

### INDUSTRIAL APPLICABILITY

The present invention is suitable for use as a medical material which is set in a catheter for treatment of a defect hole formed in a biological tissue and is particularly preferable in that the medical material is capable of being released and placed at a treatment site, enables low invasive treatment, and has almost no fear of long-term failure even when the medical material remains in the body.

### REFERENCE SIGNS LIST

100, 400 MEDICAL MATERIAL (CLOSURE PLUG)
110 FIRST CYLINDER PART
112 FIRST END PART
120 SECOND CYLINDER PART
122 SECOND END PART
130 SUBSTANTIALLY CENTRAL PART
140 COIL SPRING
150 BIOABSORBABLE FIBER
160 OUTER CYLINDRICAL PART
200 HEART
250 ATRIAL SEPTUM
252 DEFECT HOLE
300 CATHETER

1. A medical material that is formed by a cylindrical body having a stitch-like structure using a filamentary material,
the medical material
having such a shape that a cylinder diameter of a substantially central part of the cylindrical body is smaller than cylinder diameters of other parts,
being formed with a first cylinder part on a side of a first end part in a cylindrical body lengthwise direction of the medical material and a second cylinder part on a side of the other end part, with the substantially central part as a center,
comprising an elastic member both ends of which are respectively engaged with the filamentary material at the first end part and the filamentary material at the second end part, the elastic member being passed through inner parts of the first cylinder part and the second cylinder part from the side of the first end part to the side of the second end part via the substantially central part, and
being provided with an outer cylindrical body at an outermost layer of the medical material so as to cover a whole of the cylindrical body having the stitch-like structure from an outer side,
the outer cylindrical body being a cylinder-shaped body obtained by knitting a filamentary material same as or different from the filamentary material, and
both ends of the outer cylindrical body being respectively joined to both ends of the cylindrical body.

2. The medical material according to claim 1, wherein the outer cylindrical body is joined to the cylindrical body at at least one place in addition to the both ends of the outer cylindrical body.
